(19) United States
(12) Patent Application Publication
(10) Pub. No.: US 2004/0023350 A1
(43) Pub. Date: Feb. 5, 2004

(54) Method for biological oxidation of elemental sulfur-bearing materials for sulfuric acid production

(76) Inventor: John L. Uhrie, Safford, AZ (US)

Correspondence Address: William F. Mulholland, II, Snell & Wilmer L.L.P., One Arizona Center, 400 East Van Buren, Phoenix, AZ, 85004-2202, US

(21) Appl. No.: 10/211,817

(22) Filed: Aug. 1, 2002

(51) Int. Cl. C12P 3/00

(52) U.S. Cl. 435/169

(57) ABSTRACT

The present invention relates generally to a method of manufacturing sulfuric acid from elemental sulfur-bearing materials using biological oxidation processes and optimizing such processes by controlling temperature, aeration, and the biological oxidation rates of the sulfur-containing reaction solution.

### FIELD OF THE INVENTION

The present invention relates generally to a method of manufacturing sulfuric acid, and more particularly, to a method for manufacturing sulfuric acid from elemental sulfur-bearing materials using biological oxidation processes.

### BACKGROUND OF THE INVENTION

Sulfuric acid is used in a wide variety of commercial settings. For example, in connection with mining operations, sulfuric acid is used in “heap” or run-of-mine stockpile leaching of ore materials and recovery of desired metal values utilizing solvent extraction and electrowinning.

The sulfuric acid supply for use in heap and run-of-mine stockpile leaching of copper, and other base metals and/or sulfide operations can be obtained from a variety of sources, for example, as follows:

(1) smelting, with the resulting gas stream processed in an acid plant to convert SO₂ to SO₃ and subsequent production of H₂SO₄ by absorption;

(2) roasting, with gas stream processed in an acid plant to convert SO₂ to SO₃ and subsequent production of H₂SO₄ by absorption;

(3) hydro-chemical oxidation of sulfide minerals to sulfuric acid directly in solution in a sulfur burner;

(4) combustion of elemental sulfur in a sulfur-burner to produce SO₃ and subsequent production of H₂SO₄ by absorption; and/or

(5) purchase from an off-site source.

However, there are significant costs associated with the production, purchase, transfer and transportation of acid that is generated by any of these processes. Some of these processes have the ability to generate electrical energy as a by-product, which can offset the costs of acid production. However, in addition to the cost, the amount of acid required for heap and stockpile leaching operations varies with time depending on the availability of heap and stockpile feed materials. In general, this demand has been increasing world-wide in recent years.

In addition, permitting and regulatory and environmental requirements contribute to processing cost and complexity, and the application of these technologies may, in some cases, be prohibitive.

While various biological oxidation methodologies are known, such methodologies have uniformly heretofore been used in connection with metal recovery processing techniques.

Accordingly, a method to produce low-cost sulfuric acid in an environmentally acceptable manner, such as through the use of biological oxidation processing, would be advantageous.

### SUMMARY OF THE INVENTION

The present invention addresses the shortcomings of the prior art by providing a convenient and cost effective method of sulfuric acid production. While the way in which the present invention provides these advantages will be described in greater detail below, in general, elemental sulfur or elemental sulfur-bearing materials are oxidized biologically under appropriate circumstances to produce sulfuric acid. Such circumstances include, among other things, controlling temperature, aeration, and the biological oxidation rates of the sulfur-containing reaction solution. Further, a method for separating acid-containing solution from unreacted sulfur-bearing solids is also provided.

In accordance with an exemplary embodiment of the present invention, a method for manufacturing sulfuric acid from elemental sulfur-bearing materials generally includes the steps of: (i) providing a suitable elemental sulfur-bearing material; (ii) providing a biological material capable of at least partially bio-oxidizing the elemental sulfur of the elemental sulfur-bearing material; (iii) subjecting the elemental sulfur-bearing materials to biological oxidation by the biological materials; and (iv) recovering sulfuric acid from the biooxidized solution.

Advantages of a process according to various aspects of the present invention will be apparent to those skilled in the art upon reading and understanding the following detailed description with reference to the accompanying drawings.

### BRIEF DESCRIPTION OF DRAWINGS

The subject matter of the present invention is particularly pointed out and distinctly claimed in the concluding portion of the specification. A more complete understanding of the present invention, however, may best be obtained by referring to the detailed description and claims when considered in connection with the drawing figures, wherein like numerals denote like elements and wherein:

FIG. 1 illustrates a flow diagram of a method in accordance with an exemplary embodiment of the present invention; and,

FIG. 2 illustrates a flow diagram of further processing in accordance with the embodiment of the present invention illustrated in FIG. 1.

### DETAILED DESCRIPTION OF EXEMPLARY EMBODIMENTS OF THE INVENTION

In accordance with various aspects and embodiments of the present invention, a method for producing sulfuric acid from elemental sulfur-bearing materials is provided. As previously noted above, in general, the various embodiments of present invention are well-suited for use in the mining industry. Specifically, the present invention addresses the need for a sulfuric acid source that can be conveniently and economically produced in proximity to mining operations. Of course, use of the methods of the present invention are not so limited, and thus may find use in any application where sulfuric acid is needed which is now known or hereafter devised by those so skilled in the art.

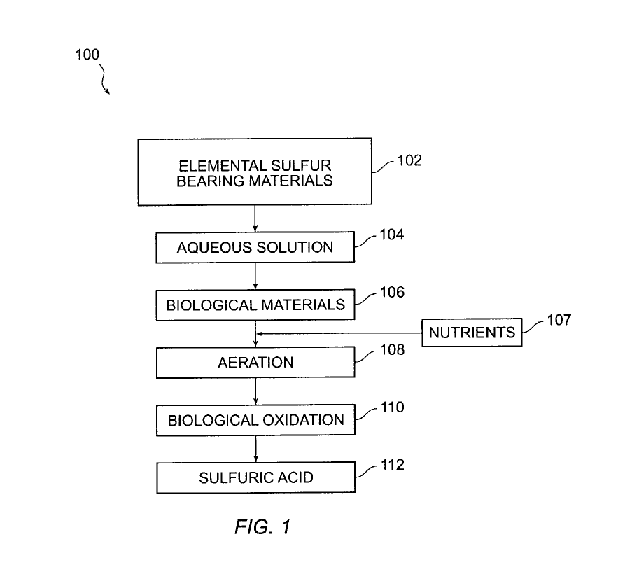

A process 100 in accordance with various embodiments of the present invention for producing sulfuric acid is generally illustrated in FIG. 1. In general, as will be described in greater detail hereinbelow, process 100 involves the formation of a suitable biological oxidation environment to which materials to be oxidized are suitably added. Preferably, the reaction materials are combined in any suitable production vessel (not shown) which facilitates biological oxidation of the materials to be oxidized. In this regard, although generally beyond the scope of this application, reference is made to co-pending application U.S. Ser. No. ______, filed on ______, entitled “Apparatus for Processing Sulfur-Bearing Materials to Produce Sulfuric Acid and Methods of Using the Same”, which discloses various suitable production vessels. By this reference the subject matter of that application is hereby incorporated herein.

With continued reference to FIG. 1, in accordance with various embodiments of the present invention, in process 100, an elemental sulfur-bearing material (step 102) is suitably provided. In the context of the present invention, the term “elemental sulfur-bearing material” refers to elemental sulfur, elemental sulfur together with other materials, or other elemental sulfur-bearing materials, such as other materials including some amount of elemental sulfur, such as some by-products of other metal recovery processes.

In accordance with another embodiment of the present invention, the term “elemental sulfur-bearing materials” also refers to other sulfur-bearing materials, such as acid generating sulfide sulfur-bearing materials including, for example, iron sulfides either alone or in conjunction with elemental sulfur or elemental sulfur-bearing materials.

In accordance with a further embodiment of the present invention, various combinations of elemental sulfur together with other materials may be provided. As a non-limiting example, such combinations may include elemental sulfur together with any other sulfides and/or other metals that might be attendant to or part of such elemental sulfur compositions.

In one exemplary embodiment, elemental sulfur-bearing material 102 comprises an elemental sulfur-containing residue produced in connection with the pressure leaching, particularly at low to medium temperatures (e.g. 85 to about 180° C.), of copper-containing material feed streams. As explained in greater detail in U.S. Ser. No. 09/915,105, such copper-containing materials include copper sulfide ores, such as, for example, ores and/or concentrates containing chalcopyrite (CuFeS₂) or mixtures of chalcopyrite with one or more of chalcocite (Cu₂S), bornite (Cu₅FeS₄), and covellite (CuS). The elemental sulfur-containing residues that result from the pressure leaching of such copper-containing material feed streams may advantageously be processed in accordance with the various aspects of the present invention.

In another exemplary embodiment, elemental sulfur-bearing material 102 comprises acid generating sulfur bearing materials, such as iron sulfides or materials containing iron sulfides or other sulfide sulfur containing materials.

For purposes of this disclosure, in most instances, the term “elemental sulfur” is used interchangeably with the term “elemental sulfur-bearing material,” inasmuch as, as will be clear from the following disclosure, the elemental sulfur and sulfide sulfur components of any sulfur-bearing material are advantageously converted to sulfuric acid in accordance with the present invention.

Elemental sulfur-bearing material 102 may be provided in any suitable form. In this regard, and in accordance with various embodiments of the present invention, elemental sulfur-bearing material 102 may be prepared for processing prior to use. For example, elemental sulfur-bearing material 102 may be prepared for processing in any manner that enables the conditions of elemental sulfur-bearing material 102, such as, for example, composition and component concentration, to be suitable for processing in accordance with the various embodiments of the present invention. That is, such conditions may affect the overall effectiveness and efficiency of processing operations. Desired composition and component concentration parameters can be achieved through a variety of chemical and/or physical processing stages, the choice of which will depend upon the operating parameters of the chosen processing scheme, equipment cost and material specifications. For example, elemental sulfur-bearing material 102 may undergo comminution, flotation, blending, wetting and/or slurry formation, as well as chemical and/or physical conditioning.

In accordance with one exemplary aspect of this embodiment of the present invention, elemental sulfur-bearing material 102 is prepared for processing by comminuting material 102 in any manner followed by suitable wetting operations. For example, in the case where elemental sulfur-bearing material 102 comprises elemental sulfur pellets of conventional form and configurations, such pellets are advantageously ground and wetted to enable the conditions of such pellets comprising elemental sulfur-bearing material 102 to be suitably processed in accordance with the present invention. Other processing techniques, including suitable feed source selection techniques, for example, to ensure that elemental sulfur-bearing materials are substantially free of biocides or other materials which may inhibit biological oxidation of material 102, may also be employed. In some applications, for example, where elemental sulfur is provided in dry powder form, further processing and/or feed source selection may not be necessary.

Referring still to FIG. 1, preferably, and in accordance with various aspects of one embodiment of the present invention, production process 100 involves combining the processed elemental sulfur-bearing materials with an aqueous solution (step 104). The aqueous solution may comprise any material capable of supporting appropriate reaction conditions for biological oxidation (“bio-oxidation”) of the elemental sulfur-bearing materials. Preferably, the aqueous solution comprises water and/or other leaching process solutions. For example, such other leaching process solutions may comprise raffinate, that is the residual solution following copper extraction in a solution extraction (or “SX”) system. In accordance with one aspect of this embodiment of the present invention, aqueous solution 104 comprises a mixture of water and raffinate.

With continued referenced to FIG. 1, conversion of elemental sulfur-bearing materials 102 to sulfuric acid in accordance with the present invention is facilitated by subjecting such elemental sulfur-bearing materials 102 to biological oxidation (step 110) utilizing an effective biological culture, comprising suitable biological materials, for example, bio-oxidizing bacteria.

As those skilled in the art will understand, the oxidation of elemental sulfur and resulting production of sulfuric acid is represented by the following reaction:

(elemental sulfur) 2S^{o}+30₂+2H₂0→2H₂SO₄ (1)

Similarly, sulfur-bearing materials, such as pyrite, are converted to sulfuric acid as follows:

(pyrite) 2FeS₂+7O₂+2H₂0→2FeSO₄+2H₂SO₄ (2)

In accordance with the present invention, utilization of bio-oxidizing bacteria enhances the oxidation rate of the sulfur-bearing materials thereby enhancing the yield and/or rate of sulfuric acid production. These advantages, in accordance with other features of the present invention, provide a method for producing sulfuric acid on a commercial scale at economically acceptable rates.

In accordance with various embodiments of the present invention, any bacteria which serve to facilitate such conversion reactions may be used to form an effective biological culture to facilitate biological oxidation step 110. The following bacteria are exemplary:

Group A: Acidithiobacillus ferrooxidans; Acidithiobacillus thiooxidans; Acidithiobacillus organoparus; Acidithiobacillus acidophilus; Acidithiobacillus caldus

Group B: Sulfobacillus thermosulfidooxidans; Sulfolobus sp.

Group C: Sulfolobus acidicaldarius; Sulfolobus BC; Sulfolobus solfataricus; and Acidianus brierleyi and the like.

These bacteria are generally available, for example, from American Type Culture Collection, or like culture collections, or are known in the art.

Alternatively, such bacteria may be naturally occurring and obtained and cultured, or otherwise grown in any conventional, now known, or hereafter devised method. For example, in certain applications, naturally occurring biological strains may be used. In accordance with one preferable aspect of this embodiment, mixed bacterial strains occurring naturally in raffinate streams may be initially added to the aqueous solution and allowed to undergo a natural selection process. Such selection processes may involve, among other things, the reaction environment. It has been found that such naturally occurring bacterial strains may be particularly useful in connection with applications of the present invention in connection with mining activities. However, bacterial strains may be selected by any technique now know or developed in the future; however, in accordance with an exemplary embodiment of the invention, bacterial strains may be selected from the list provided above.

These bacteria may be classified in terms of their temperature tolerances and optimized growth and activity ranges as follows: mesophiles, moderate thermophiles, and extreme thermophiles. Mesophilic bacteria generally thrive under moderate operating temperatures (less than 40° C.); moderate thermophiles are generally optimized for higher temperature conditions (37-60° C.); and, extreme thermophiles generally thrive at more extreme temperatures (e.g., exceeding 55° C.). Group A bacteria are generally considered mesophilic and are preferably operated under conditions at or below 40° C.; Group B bacteria is representative of the moderate thermophile type and is preferably operated at less than 60° C.; and, Group C bacteria is representative of the extreme thermophile group and is preferably operated under conditions between 60-800C. Various mixtures of bacteria from various groups can also be obtained.

In accordance with a preferred aspect of the present invention, Acidithiobacillus caldus bacteria are utilized under operating conditions at or about 40° C. For example, a suitable biological environment has been prepared by collecting and culturing mine water containing such bacteria in a conventional manner. For example, appropriate biomass production may be practiced by techniques commonly known in the art, such as disclosed in “Biology of Microorganisms,” Madigan and Marttinko, Ninth Ed., Prentice-Hall (2000). In accordance with the present invention, a biomass concentration on the order of about 1×10⁹ cells per milliliter is preferred. However, any bacteria selection and growth processes now known or developed in the future may be used in accordance with the present invention. Furthermore, as noted above, any bacteria which facilitate the convenient and efficient production of sulfuric acid may be used. Moreover, the listings of bacteria and temperature-based classifications set forth herein are provided for illustration only, and are not in any way limiting of the bacteria that may be used in accordance with the present invention. Any biological material including microbial agents, microorganisms, bacteria, and the like, which are capable of at least partially oxidizing sulfur containing materials, may be used in accordance with the methods herein described.

Preferably, the biological culture is provided to any suitable production vessel to facilitate biological oxidation (step 110) of the elemental sulfur-bearing materials 102 to sulfuric acid, where sulfuric acid is then recovered (step 112). Biological oxidation 110 preferably is facilitated in any conventional, now know, or hereafter devised manner. For example, and with continued reference to FIG. 1, during biological oxidation, air or another oxygen-containing source (step 112) is suitably supplied to the production vessel, as may be needed, in addition to other materials, including nutrients, biological materials or other additives, such as wetting agents, processing aids and the like.

As will be discussed further below, preferably, it is desirable to establish a substantially self-sustaining bacteria population to facilitate biological oxidation step 110. The sustainability of such populations may be promoted by adjusting various parameters of the reaction environment, including, among others, controlling temperature, aeration, and nutrient addition.

Aeration (step 112) is preferably initiated before addition of elemental sulfur-bearing materials 102. In a preferred embodiment, aeration 112 commences prior to the addition of elemental sulfur-bearing materials 102. More preferably, aeration 112, once commenced, proceeds continuously until such time as it becomes desirable to terminate the sulfuric acid production processes and/or until such time as viable bacteria populations within the aqueous solution are no longer desired.

As will be appreciated, aeration (step 112) provides oxygen to the solution. In general, oxygen delivery requirements are a function of, among other things, the oxygen requirements for optimized bacterial growth and activity as well as the oxygen requirements for the sulfur oxidation reaction. The amount of oxygen dissolved in the solution may affect the rate of sulfur oxidation. For example, in general, the oxidation rate increases as the dissolved oxygen increases, up to a value where the mass transfer of oxygen is no longer rate determining. The exact value of this requirement is dependent upon many factors including the concentration of solids dissolved in solution, bacterial population and activity, temperature, agitation, and other solution conditions.

The amount of dissolved oxygen also affects the active state of bacteria. For example, after reaching the active bio-oxidation stage of its life cycle, bacteria may lapse into a dormant stage or die if oxygen concentrations fall below a critical value. From this phase, bacteria may be slow to recover once higher oxygen concentrations are subsequently restored.

Air delivery into the aqueous medium within which biological oxidation step 110 takes place is also a function of the oxygen uptake rate. The oxygen uptake rate is a measurement of the rate at which oxygen is required to maintain a given concentration of oxygen in solution. This uptake rate, in turn, is generally dependent upon the oxygen utilization factors described above, namely desired oxidation rates and bacterial activity.

Furthermore, air delivery requirements should be determined by calculating the oxygen content of ambient air plus a utilization factor. In general, this factor ranges from about 10 to about 40%, and typically is on the order of about 30%.

Therefore, it is preferable, in accordance with various aspects of the present invention, that dissolved oxygen concentrations are provided and maintained at suitable minimum levels so as to promote biological activity and sulfur oxidation. That is, preferably, aeration step 112 maximizes air delivery and oxygen transfer rates, within acceptable economic limits, but at sufficient levels to facilitate and maintain oxidation of elemental sulfur-bearing materials 102. Preferably, minimum dissolved oxygen concentrations should be between about 2.5 to 4.0 mg/l and preferably about 4.0 mg/l. Higher dissolved oxygen concentrations can be utilized but generally are not economically viable. In any event, maximum levels should not exceed levels toxic to biological materials.

However, one skilled in the art will recognize that oxygen uptake requirements will vary depending upon various reaction conditions, including percent solids in solution, bacteria, temperature, elevation, and reactor vessel design, among others, and that other suitable oxygen levels to facilitate oxidation should be utilized.

Preferably, aeration step 112 comprises utilization of any suitable surface or subsurface discharge device for providing discharge of air into the aqueous media. If a reactor vessel is used, an air source may be positioned in association with the lower portion of the vessel to facilitate diffusion through the bulk of the solution as the oxygen migrates in an upward fashion toward the surface. As those skilled artisans well appreciate, a variety of different sparging systems, reactor types, or other air delivery devices are known. As such, any air delivery device that is now know, or hereafter devised that can be suitably configured to facilitate delivery of air into the aqueous media to facilitate biological oxidation step 110 may be used.

In accordance with various aspects of the present invention, elemental sulfur-bearing materials 102 are subjected to biological oxidation (step 110), and during such biological oxidation, various materials are added, as necessary, to facilitate, enhance or otherwise control the biological oxidation processes. Bio-oxidation preferably proceeds such that at least some, and more preferably a substantial portion of the elemental sulfur-bearing materials are suitably converted to sulfuric acid 112. As will be discussed in greater detail hereinbelow, in accordance with one embodiment of the present invention, biological oxidation preferably proceeds to substantially oxidize a majority of the initially provided elemental sulfur-bearing material. In such cases, and in accordance with various aspects of one embodiment of the present invention, with continued reference to FIG. 1, additional biological materials (step 116), nutrients (step 118) or other materials (not shown) may be advantageously added in suitable amounts and at suitable times during biological oxidation 110. Such biological materials may include any of the aforementioned bacteria or other biological materials, bacteria containing materials from other production vessels (in the case of staged or other systems described in greater detail hereinbelow), or any other biological material which may facilitate biological oxidation of elemental sulfur-bearing materials.

In accordance with further aspects of this embodiment of the present invention, as briefly noted above, the process 100 optionally may include the addition of nutrients (step 118). Though bio-oxidizing biological materials, including bacteria, derive energy, in part, from the oxidation of sulfur, additional nutrient materials may aid in cell growth and oxidation functions.

Nutrients, including ammonia, phosphate, potassium, and magnesium may be added to facilitate oxidation processes and aid cell growth and maintenance. For example, these nutrient constituents may be introduced in any suitable media, including a Modified Kelly's Media (MKM), in the following concentrations comprising:

| | | |
|---|---|---|
| (NH₄)₂SO₄ | (0.4 | gpl) |
| K₂HPO₄ | (0.04 | gpl) |
| MgSO₄ 7H₂O | (0.4 | gpl) |

However, other nutrient constituents and concentrations may be used, depending on the precise requirements and conditions of the desired system. For example, the nutrient constituents of ambient air, such as carbon dioxide, may also be used to enrich the reaction media. Other forms of enriched air may also be used in accordance with the present invention, including, for example, enriched carbon dioxide and enriched oxygen air. However, enrichment of the reaction media may proceed by any other suitable method, now known or developed in the future.

Bio-oxidation rates are subject, in part, to the rate limiting conditions described above, including oxygen mass transfer and sulfur substrate availability. In addition, induction times for bio-oxidizing activity, growth cycles, biocide activities, bacteria variability, and the like, as well as economic considerations, all affect the rates and duration of bio-oxidation (step 110) in accordance with the exemplary embodiments.

In accordance with various embodiments of the present invention, either continuous or batch-type biological oxidation systems may be used. For example, in a batch-type system reaction conditions are established and proceed for a limited time or finite duration. For example, as illustrated in FIG. 1, constituent reaction components are combined, such as the elemental sulfur-bearing materials (step 102), in a reaction environment, such as a reaction vessel, and biological oxidation processes.

However, such a batch system may require a significant induction period before commercially acceptable rates of conversion proceeds. This is due, in part, to the four stages typically associated with bacteria life cycles as follows: (1) the lag stage; (2) the logarithmic growth stage; (3) the active stage; and (4) the death or dying stage. Depending upon the mineralogy of the sulfur-bearing materials and solution conditions, such as dissolved oxygen concentration, temperature, and the like, this period may last from 2 to 3 days to several months. Generally, the greater the change in environmental conditions for the bacteria, the greater the time required for the induction period. In any event, in accordance with this embodiment, bio-oxidation 110 will proceed such that the sulfur-bearing materials are subjected to biological oxidation to produce a solution with suitable concentrations of sulfuric acid (step 112). For example, the bio-oxidation process may proceed until desired level of sulfur conversion is achieved, or until the biological materials no longer exhibit the desired level of activity. The resulting sulfuric acid solution (step 112) is then recovered (step 114) and used for various applications.

However, preferably, in accordance with various other aspects of the present invention, a continuous system is used. In certain aspects of this embodiment of the present invention, multiple stages are employed to obtain substantial oxidation; in others, substantial oxidation is obtained in a single biological oxidation step. Preferably, in continuous systems, various reaction conditions are maintained approximately at a steady state, such as, for example, where a continuous supply of sulfur-bearing materials are continually subjected to biological oxidation resulting in a regular supply of sulfuric acid. In an exemplary embodiment, establishment of reaction conditions proceeds approximately as outlined in FIG. 1. Elemental sulfur (step 102) is added to an aqueous solution (step 104) and provided to a production vessel that is subjected to aeration (step 108), the provision of nutrients (step 118), and optionally, as needed, the provision of additional biological materials (step 116). After an appropriate induction period, bio-oxidation (step 110) then proceeds where the elemental sulfur-bearing materials are subjected to biological oxidation to produce a solution with suitable concentrations of sulfuric acid (step 112).

Preferably, a regular supply of influent is continuously delivered into the reactor vessel and a regular supply of effluent may be continually recovered therefrom. For example, the influent may include sulfur-bearing materials, water, and/or other process leaching solutions, nutrients, additional biological materials, and/or other components. The effluent preferably comprises a dilute sulfuric acid solution, but preferably excludes unreacted or partially reacted sulfur-bearing materials. In this manner, bio-oxidation conditions may be kept at approximately a steady state where the amount of sulfur-bearing materials entering the system is approximately equal to the sulfuric acid eluted therefrom.

However, in accordance with various aspects of a preferred embodiment of the present invention, processing is conducted in a manner which permits decoupling of the solid and liquid retention rates. Stated another way, preferably, the retention time of the liquid within the production vessel is much less than the retention time of the solids. As will be apparent from the description that follows, various advantages may be obtained from this decoupling; for example, the production vessel(s) can be appropriately sized while acid production rates can be maximized.

In general, retention time can be conveniently defined as the production vessel volume divided by the flow rate, and is typically expressed in hours. Similarly, the liquid (e.g., solution) retention time can be conveniently expressed as the production vessel volume divided by the liquid flow rate. As such, the liquid retention time is a measure of the length of time an average liquid particle is retained within the production vessel. On the other hand, the solids retention time can be conveniently expressed as the production vessel volume divided by the solids flow rate. Thus, the solids retention time is a measure of the length of time an average solid particle is held within the production vessel.

The present invention advantageously enables solid retention times to be significantly longer than liquid retention times. Such retention times are, however, influenced by the amount of solids provided to and maintained in the production vessel, that is the percent of solids which are contained in the production vessel percent solids), as well as the desired acid concentration levels of the produced sulfuric acid.

In general, depending on its intended use or application, the acid concentration may be varied as desired. However, acid concentration levels exceeding the tolerance levels of the selected biological materials, in general, should be avoided, for obvious reasons. Typical biological materials have tolerance levels in the range of about 35 gpl acid. Accordingly, acid concentration levels, that is the acid concentration of the sulfuric acid produced in accordance with the present invention, is preferably less than about 35 gpl. Where the reaction conditions permit, however, higher acid levels may be obtained and permitted.

In general, the percent of solids provided to the production vessel is related to the oxidation rate of the solids, for example per unit volume added, as well as the production vessel volume. Preferably, the percent solids maintained in the production vessel can be in the range of about 5 to about 30%, but more preferably is selected to be in the range of about 15 to about 22%, and optimally in the range of about 16 to about 20%. In any event, preferably, the percent solids is selected to maximize the acid production rate.

Inasmuch as the oxidation rates may vary depending on the percent solids in the production vessel, the concentration of the acid produced may also vary depending on the percent solids selected.

As briefly noted above, the decoupling of the liquid retention time from the solid retention time enables suitable parameters to be economically obtained. In general, such decoupling can be obtained in any now known or hereafter devised technique. For example, reference is made to the previously referenced co-pending application, namely U.S. Ser. No. ______. However, preferably, the elemental sulfur-bearing materials are suitably retained and substantially oxidized within the production vessel in accordance with the present invention.

For example, in accordance with one aspect of the present invention, a solid/liquid separation device may be used in association with the reactor vessel. In this regard, generally, any solid/liquid separation device may be used in accordance with this aspect of the present invention including diffusers, settlers, screeners, thickeners, clarifiers, and preferably elutriators or any other device suitably retains the unreacted or partially reacted solids within the production vessel.

In accordance with various preferred aspects of this embodiment of the present invention, the solids retention time, is suitably maintained to permit sufficient conversion, and thus optimize sulfuric acid yield. In general, the greater the residence time, the greater the recovery yield of sulfuric acid. However, this increased yield may be offset by the corresponding increase in processing costs for each incremental increase in additional processing time. Preferably, the solids retention time is on the order of at least thirty (30) days, and generally is between about 30 to about 60 days.

On the other hand, the liquid retention times obtainable through use of the present invention are typically on the order of up to about 1.5 to about 5 days, and preferably on the order of between about 0.5 to about 3 days. More preferably, liquid retention times are on the order of between about 1 to about 2 days, and optimally on the order of between about 1 to about 1.5 days.

Preferably, in accordance with various aspects of the present invention, in continuous systems, liquid retention time determinations should also account for the regeneration time of biological materials. Preferably, the biological reaction environment, including biological materials, such as bacteria, should be maintained in a logarithmic growth stage. Accordingly, it is preferable to select a liquid retention time so that it exceeds the doubling time of the bacteria. The doubling time of the biological materials refers to the time required for a given amount of biological materials to reproduce in equal number. For preferred biological materials, typically this doubling time is on the order of 1 day. In any event, preferably, the liquid retention time is selected to be greater than or equal to the doubling time of the bacteria. In this manner effective use of the biological materials is ensured.

The following example illustrates various advantages of the present invention.

### EXAMPLE

A single agitated production vessel, having a volume of 784(l) and including a conventional solid/liquid separator was provided. The separator was operatively connected to the production vessel to suitably remove effluent containing solids and liquid. A sulfur feed, that is elemental sulfur feed, was suitably prepared and provided to the vessel at a rate of 3.1 kg/day. In addition, an aqueous feed of MKM solution, principally comprising water, biological nutrients, and sulfuric acid at O gpl acid, was also added to the vessel at a rate of 525 l/day. In so doing the percent solids in the vessel was maintained at about 20%. The vessel was agitated and biological oxidation commenced. Material was continuously withdrawn from the vessel and passed through the solid/liquid separator. The solids were suitably returned to the vessel.

The liquid, sulfuric acid, was separated and recovered. It was determined to have an average acid strength of 17.4 g/l H₂SO₄, or corresponding to 9.1 kg H₂SO₄/day.

Analysis of the system demonstrated a % conversion of 95% of elemental sulfur to sulfuric acid. Solid (e.g., sulfur) retention times were determined to be 1356 hours (56.5 days). Liquid (solution) retention times were determined to be 32 hours (1.3 days).

The foregoing example demonstrates that by decoupling the solid and liquid retention times, that is by mechanically retaining the elemental sulfur in the production vessel, suitable conversion rates of desirable acid could be effectively obtained.

It should be appreciated that any number of vessels can be advantageously used in accordance with the present invention and its various embodiments.

For example, with reference to the aforementioned example, if the solid and liquid are permitted to flow concurrently from one vessel to the next with elemental sulfur and liquid feeds being the same, conversion rates being the same, and withdrawn acid being the same, and other operating conditions being the same, up to 38 vessels would be needed to effectively provide similar solids retention and replicate the results.

While such systems may be employed, the economic consequences are clear. However, various modifications may be made wherein some or all of the production vessels are configured to retain in whole or in part the unreacted or partially reacted solids.

For example, in accordance with a further embodiment of the present invention, one or more production vessels may be positioned in a down-stream relationship from a primary production vessel for continued processing of the unreacted sulfur-bearing materials. Processing in each production vessel proceeds generally in a manner similar to the steps outlined in FIG. 1; however, in this embodiment, aqueous solution introduced into the downstage reactor vessel may contain, among other things, unreacted elemental sulfur, and biological materials from previous reactor vessels. Accordingly, admixture of additional elemental sulfur-bearing materials and biological materials is suitably selected to take these factors into account.

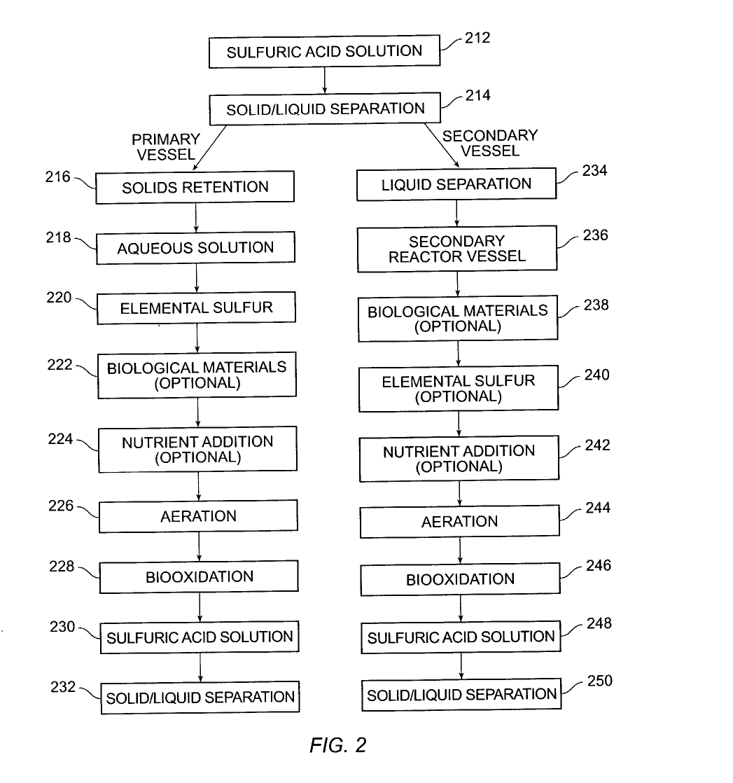

For example, and with reference now to FIG. 2, a process for sulfuric acid production using primary and secondary vessels is shown. As such, sulfuric acid solution 212 is formed in a primary reactor vessel similar to the process described with reference to FIG. 1 hereinabove. Sulfuric acid solution 212 is then subjected to solid/liquid separation (step 214). Preferably such separation enables bulk solids, for example, unreacted elemental sulfur-bearing materials to be retained in the primary reactor vessel (step 216), while the liquid separation, for example sulfuric acid, is separated therefrom and transferred to a secondary reactor vessel (step 236).

Solids retained in the primary reactor vessel (step 216) are suitably subjected to further processing. For example, additional influent may be added to the primary reactor vessel and comprise various reaction constituents, including additional aqueous solution (step 218), additional elemental sulfur-bearing materials (step 220), additional bacteria, or combinations thereof, as needed. Aeration (step 226), as discussed above, preferably, occurring continuously, enables further bio-oxidation of the elemental sulfur-bearing materials to yield sulfuric acid (step 230). Further solid/liquid separation (step 232) may be effected to repeat the process over again.

As briefly described above, and in accordance with a further aspect of this embodiment of the present invention, the bulk solution, that is sulfuric acid, eluted from the primary reactor vessel is suitably transferred to another reactor vessel (step 236) and subjected to further processing as further illustrated in FIG. 2. Liquid solution entering the secondary reactor vessel may contain unreacted sulfur-bearing solids in addition to biological materials and sulfuric acid. Aeration (step 244), and preferably on a continuous basis, continues and bio-oxidation (step 246) proceeds, generally as described above, such that the elemental sulfur-bearing materials are subjected to further biological oxidation by biological materials. Additionally, depending upon the established reaction conditions, it may also be desirable to add additional biological materials (step 238) and/or elemental sulfur-bearing compounds (step 240).

The resulting sulfuric acid solution (step 248) in the secondary vessel is generally more highly concentrated relative to the sulfuric acid solution (step 230) generated in the primary reactor vessel. Thus, through subsequent processing stages a more highly concentrated sulfuric acid may be obtained, and substantially complete oxidation of the elemental sulfur-bearing materials to sulfuric acid facilitated.

After sulfur-bearing materials have been suitably converted in accordance with the various aspects of the various embodiments of the present invention, sulfuric acid is recovered from the reactor vessel (step 114) and collected in an appropriate manner. Influent and effluent flow rates may be suitably adjusted in accordance with desired residence times, among others, as discussed above. In accordance with the present invention, this flow may be continuous or intermittent. The composition of the effluent preferably contains sulfuric acid with only minor amounts of unreacted or unoxidized sulfur-bearing materials. The effluent may also contain bacteria nutrients, and other constituent components of the reaction solution.

Effluent acid concentrations will vary according to residence times, percent solids, bacteria concentrations, and other factors discussed above. In the above-described exemplary two-stage reactor circuit, the acid concentration of primary stage effluent may be on the order of from 15 to about 25 g/l acid; second stage effluent on the order of from about 20 to about 30 g/l acid.

The recovered sulfuric acid may be used in any desired manner. For example, it may be introduced into a raffinate stream for later use in leaching processes. Alternatively, the recovered acid may be subjected to further processing (step 116) to achieve higher acid concentrations in the range of about 100 to about 300 g/l of acid by any suitable method known in the art, including ion exchange processes or other suitable processes. These and other uses as are now known or as of yet are unknown but may be later developed by those skilled in the art may be made of the sulfuric acid conveniently and effectively produced in accordance with the present invention.

Certain features may be added or adjusted to optimize sulfuric acid production and/or recovery in accordance with various embodiments of the present invention. These include: (1) oxygen dispersion; (2) agitation; (3) temperature control; (4) or other circuit design enhancements, and the like.

In accordance with various aspects of the present invention, not only is it advantageous that oxygen be delivered into the reaction mixture, but also that it be provided in a form that encourages efficient dispersion into solution.

In accordance with one aspect of the present invention, air is introduced as finely dispersed air bubbles into solution in order to maximize the surface area and mass transfer rates. For example, air may be discharged into a diffuser positioned in association with the aqueous media. Diffusers such as a porous rock, grated mesh or openings, and/or similar devices may be utilized for such purposes.

Oxygen dispersion may also be encouraged by a shearing device provided to shear the air bubbles into smaller particles in solution thereby enhancing the surface area of the air bubbles, which facilitates greater dispersion into solution. A preferable shearing device is an impeller positioned in association with the air discharge point.

Commercially available impellers configured to promote air dispersion may be used and can be employed in a variety of reactor vessels. Impeller tip speed is limited, in part, by the potential shearing effects on the bacteria in solution. In this regard, a tip speed range of about 2 to about 4 m/sec is preferred, and more preferably tip speed ranges on the order of about 2 to about 3 m/sec. The shearing impeller is preferably placed in proximity to the air discharge point at any suitable distance from the bottom of the reaction vessel. Placement, of course, may vary depending upon the particular application, but, in general, should be selected to maximize, or at least enhance the air/oxygen surface area, and to minimize diffusion of the air/oxygen into the reaction mixture prior to such action.

In addition to dispersing the air introduced into the reactor vessel, in certain instances, it is desirable to agitate and/or blend the aqueous media. In an exemplary embodiment, sufficient agitation may be accomplished by the shearing device. However, in other cases, one or more additional impellers may be used to facilitate further agitation and/or mixing of the reaction solution and thereby facilitate diffusion of air existing within the headspace of the reactor vessel about the surface of the aqueous solution.

Due to its hydrophobic properties, sulfur, for example elemental sulfur, may collect on top of the reaction solution as a froth. Accordingly, in some cases it may be preferable to position a mixing impeller near the surface of the reaction solution in order to agitate or chum the top layer of solution to facilitate wetting of the frothing sulfur, in addition to the other properties previously discussed.

The method of the present invention, in accordance with its various aspects and embodiments, may be influenced by temperature. Depending upon the desired specific reaction conditions and bacteria selected, the reaction temperature is preferably maintained in the range of about 35 to about 60° C. The temperature of the reaction solution may be maintained in a variety of ways. For example, the temperature of the solution itself may be controlled, or alternatively, the reactor vessel temperature. For example, a heat exchange device in association with the reactor vessel, such as a cooling/heating jacket, may be used.

The present invention has been described above with reference to a number of exemplary embodiments. It should be appreciated that the particular embodiments shown and described herein are illustrative only and are not intended to limit in any way the scope of the invention as set forth in the claims. Those skilled in the art having read this disclosure will recognize that changes and modifications may be made to the exemplary embodiments without departing from the scope of the present invention. For example, although reference has been made throughout to sulfuric acid production in a production vessel, it is intended that the invention also be applicable to any suitable configuration capable of containing an aqueous medium during bio-oxidation such as in-ground containment vessels, ponds, and the like. Further, although certain preferred aspects of the invention, such as techniques and apparatus for aeration of the aqueous solution and arrangements of production vessels in a circuit, for example, are described herein in terms of exemplary embodiments, such aspects of the invention may be achieved through any number of suitable devices now known or hereafter devised. Accordingly, these and other changes or modifications are intended to be included within the scope of the present invention, as expressed in the following claims.

1. A method for biologically producing sulfuric acid comprising the steps of:
(a) providing an elemental sulfur-bearing material;
(b) providing a suitable biological material capable of at least partially biooxidizing elemental sulfur associated with said elemental sulfur-bearing material;
(c) subjecting said elemental sulfur-bearing materials to biological oxidation by said biological materials; and
(d) recovering said sulfuric acid.

2. The method of claim 1 wherein said biological oxidation step (c) further comprises the steps of:
(a) providing a suitable aqueous solution for said elemental sulfur-bearing materials and said biological materials; and,
(b) aerating said aqueous solution.

3. The method of claim 2 wherein said step (a) of providing elemental sulfur-bearing material comprises providing elemental sulfur by-products from other metal recovery processes.

4. The method of claim 2 wherein said step (a) of providing elemental sulfur-bearing material comprises providing elemental sulfur, iron sulfides or mixtures thereof.

5. The method of claim 2 wherein said step (a) of providing elemental sulfur-bearing material comprises providing elemental sulfur residues from the pressure leaching of copper-containing feed streams.

6. The method of claim 2 wherein said step (b) of providing suitable biological materials comprises providing Acidithiobacillus caldus bacteria.

7. The method of claim 2 wherein said step (b) of providing suitable biological materials comprises providing bacteria naturally occurring in raffinate streams.

8. The method of claim 2 wherein said step (b) of providing suitable biological materials further comprises growing said biological materials in a suitable medium prior to admixture of said biological materials with said elemental sulfur-bearing material.

9. The method of claim 1 wherein said biological oxidation step (c) is conducted such that substantially all of the elemental sulfur-bearing material provided in step (b) is converted to sulfuric acid.

10. The method of claim 1 wherein said biological oxidation step (c) further comprises adding nutrients to said biological materials to aid in growth and biological oxidation activity.

11. A biological oxidation method comprising the steps of:
(a) subjecting an elemental sulfur-bearing material to biological oxidation;
(b) separating the product into a sulfuric acid fraction and a residual fraction which contains at least partially unoxidized elemental sulfur-bearing material; and
(c) recovering said sulfuric acid fraction.

12. The method of claim 11 wherein said step (a) of subjecting an elemental sulfur-bearing material to biological oxidation results in substantially complete oxidation of said elemental sulfur-bearing material to sulfuric acid.

13. The method of claim 11 further comprising the step (d) subjecting said residual fraction further biological oxidation.

14. The method of claim 11 further comprising the step of subjecting said sulfuric acid fraction to further biological oxidation prior to step (c).

15. A process for producing sulfuric acid from elemental sulfur containing materials by biooxidation comprising treating a solution containing the elemental sulfur-bearing material with bacteria capable of oxidizing said elemental sulfur of the elemental sulfur-bearing material and aerating said solution.

16. A biological oxidation method comprising the steps of:
(a) subjecting an elemental sulfur-bearing material to biological oxidation;
(b) separating the product into a liquid fraction and a solids fraction wherein said solids fraction contains at least partially unoxidized elemental sulfur-bearing material;
(c) subjecting said solids fraction to further biological oxidation; and
(d) recovering sulfuric acid from said liquid fraction.

17. The method of claim 16 wherein said step (c) of subjecting said solids fraction to further biological oxidation results in said solids fraction having a retention time longer than the retention time of said liquid fraction.

18. The method of claim 16 wherein said solids retention is from about 30 days to about 60 days.

19. The method of claim 16 wherein said liquid retention is from about 0.5 days to about 10 days.
